(19) United States
(12) Patent Application Publication
(10) Pub. No.: US 2025/0113145 A1
(43) Pub. Date: Apr. 3, 2025

(54) SOUND ENVIRONMENT CONTROL SYSTEM AND SOUND ENVIRONMENT CONTROL METHOD

(71) Applicant: Mitsubishi Electric Corporation, Tokyo (JP)

(72) Inventors: Kota KURIHARA, Chiyoda-ku, Tokyo (JP); Seiji NODA, Chiyoda-ku, Tokyo (JP); Makoto TAKATA, Chiyoda-ku, Tokyo (JP); Yoichi ITO, Chiyoda-ku, Tokyo (JP); Ayumu OSUMI, Chiyoda-ku, Tokyo (JP)

(73) Assignee: Mitsubishi Electric Corporation, Tokyo (JP)

(21) Appl. No.: 18/834,247

(22) PCT Filed: Feb. 7, 2022
(86) PCT No.: PCT/JP2022/004699

§ 371 Date: Jul. 30, 2024

(51) Int. Cl. H04R 3/12; A61M 21/00; G10K 15/04

(52) U.S. Cl. CPC H04R 3/12; A61M 21/00; G10K 15/04; A61M 2021/0027; H04R 2430/01

(57) ABSTRACT

A sound environment control system according to the present disclosure controls sound environment in a room, and includes an information processing device, an output device, and a sensor. The information processing device generates a sound having a plurality of frequency components. The output device outputs the sound generated by the information processing device into the room. The sensor senses biometric information of a person in the room. The sound includes a meaningless sound that is meaningless to the person. The information processing device determines conditions of the person, using the biometric information sensed by the sensor. Depending on the determined conditions of the person, the information processing device adjusts at least one of a frequency and a magnitude of at least one frequency component forming the meaningless sound.

### TECHNICAL FIELD

The present disclosure relates to a sound environment control system and a sound environment control method.

### BACKGROUND ART

Japanese Patent Laying-Open No. H7-59858 (PTL 1) discloses a relaxing acoustic device. The relaxing acoustic device is configured to output three types of sinusoidal audio frequency signals having a frequency difference of a few Hertz therebetween, while simultaneously outputting sound information such as music. The relaxing acoustic device can provide increased realism and an increased sense of relaxation by letting a listener listen three-dimensionally to the three types of sinusoidal audio frequency signals, as compared to letting the listener just listen to sound information such as music.

### CITATION LIST

### Patent Literature

PTL 1: Japanese Patent Laying-Open No. H7-59858

### SUMMARY OF INVENTION

### Technical Problem

Individuals have different preferences for the sound environment. Due to this, sound environment may have effects of enhancement of the realism and a sense of relaxation of one listener, while other listener may not notice such effects. Therefore, in order to provide certain effects to all listeners, it is necessary that individual preferences are previously studied and results of the studies are reflected to the control of the sound environment.

The present disclosure is made to solve such a problem, and an object of the present disclosure is to provide a sound environment control system and a sound environment control method that can provide sound environment in which people's work efficiencies or comfort can be improved, independent of individual preferences.

### Solution to Problem

A sound environment control system according to the present disclosure controls sound environment in a room in which a person is present. The sound environment control system includes an information processing device, an output device, and a sensor. The information processing device generates a sound having a plurality of frequency components. The output device output the sound generated by the information processing device into the room. The sensor senses biometric information of the person. The sound includes a meaningless sound that is meaningless to the person. The information processing device determines conditions of the person, using the biometric information sensed by the sensor. The information processing device adjusts at least one of a frequency and a magnitude of at least one frequency component forming the meaningless sound, depending on the determined condition of the person.

A sound environment control method according to the present disclosure is a sound environment control method for controlling sound environment in a room in which a person is present, the sound environment control method including generating, by a computer, a sound having a plurality of frequency components; outputting, by the computer, the generated sound into the room; and sensing, using a sensor, biometric information of the person. The sound includes a meaningless sound that is meaningless to a person. Generating the sound includes determining conditions of the person, using the biometric information sensed by the sensor; and adjusting at least one of a frequency and a magnitude of at least one frequency component forming the meaningless sound, depending on the determined conditions of the person ADVANTAGEOUS EFFECTS OF INVENTION

According to the present disclosure, the sound environment can be provided in which people's work efficiencies or comfort can be improved, independent of individual preferences.

### BRIEF DESCRIPTION OF DRAWINGS

FIG. 1 is an overall block diagram of a sound environment control system according to the present disclosure.

FIG. 2 is a diagram showing a hardware configuration of an information processing device.

FIG. 3 is a diagram showing a functional configuration of the information processing device.

FIG. 4 is a process flow diagram of a sound environment control method according to the present embodiment.

FIG. 5 is a graph showing a sound output from the sound environment control system into a room versus the work efficiency of a subject in the room.

FIG. 6 is a graph showing a sound output from the sound environment control system into the room versus the subject's brainwave state in the room.

FIG. 7 is a process flow diagram of the sound environment control method according to Variation 1 of the present embodiment.

FIG. 8 is an overall block diagram of the sound environment control system according to Variation 2 of the present embodiment.

FIG. 9 is a process flow diagram of the sound environment control method according to Variation 2 of the present embodiment.

### DESCRIPTION OF EMBODIMENTS

An embodiment according to the present disclosure will be described in detail, with reference to the accompanying drawings. Note that the same reference sign is used to refer to the same or corresponding component in the drawings, and description thereof will not be repeated.

### Embodiment 1

### <Configuration of Sound Environment Control System>

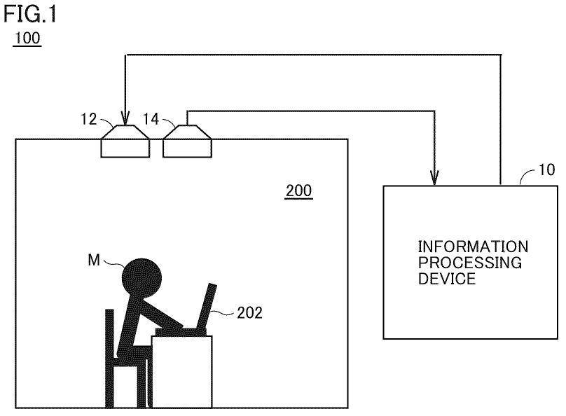

FIG. 1 is an overall block diagram of a sound environment control system according to an embodiment of the present disclosure.

As shown in FIG. 1, a sound environment control system 100 is a system for controlling the sound environment in a room 200. A person M is in the room 200. In the example of FIG. 1, the person M is engaged in input work on a terminal device 202 (e.g., a notebook).

The sound environment control system 100 includes an information processing device 10, an output device 12, and a sensor 14. The information processing device 10 is connected to the output device 12 and the sensor 14 so as to be communicable by wire or wirelessly. The information processing device 10 may be installed inside or outside the room 200. The information processing device 10 may be communicatively connected to the output device 12 and the sensor 14 via a communication network (typically, the Internet) not shown.

The information processing device 10 generates a sound that has multiple frequency components. The frequency components include at least one frequency component in an audio frequency band. The audio frequency band is a frequency range audible to humans, and is, generally, a frequency band from 20 Hz to 20 kHz. The frequency components can further include a frequency component in an ultra-high frequency band (a frequency band higher than 20 kHz) that is not audible to humans.

The information processing device 10 is configured to generate a meaningful sound and a meaningless sound. The “meaningful sound,” as used herein, refers to a sound that is meaningful to a person. Examples of the meaningful sound include music, a person's talking, and reading aloud. The “meaningless sound,” as used herein, refers to a sound that is meaningless to a person. Examples of the meaningless sound include nature sounds such as sound of sea waves, sound of wind, sounds of tree leaves rustling, and babbling brooks, sounds of traffics such as automobiles, trains, or aircrafts, street sounds, people's footsteps, and hum of air-conditioning equipment.

The information processing device 10 is configured to generate a sound which includes at least one of the meaningless sound and the meaningful sound, depending on an output of the sensor 14. This allows the sound environment control system 100 to have: a mode in which the room 200 is provided with a meaningful sound only; a mode in which the room 200 is provided with a synthetic sound obtained by synthesizing a meaningful sound and a meaningless sound; and a mode in which the room 200 is provided with a meaningless sound only. The sound environment control system 100 can alternately switch these modes.

The output device 12 is installed in the room 200, and outputs into the room 200 the sound generated by the information processing device 10. The output device 12 is, typically, a loudspeaker or a headphone. The output device 12 converts an electrical signal received from the information processing device 10 into an audio signal, and outputs the audio signal into the room 200 as a sound. While FIG. 1 shows one output device 12, it should be noted that multiple output devices may be used to output a sound into the room 200.

The sensor 14 senses biometric information of the person M in the room 200. The biometric information includes information indicating the biological conditions and information indicating body activities or movements. Examples of the biometric information include person's eye movements (ocular movements, blinking times, pupil diameter, etc.), arm (in particular, hand) movements, a pulse rate, a heart rate, brainwaves, sweating, or the temperature of peripheral body parts. Any of these biometric information can be sensed, using a well-known non-contact or contact sensor. The sensor 14 is, typically, a wearable device worn by a person or a camera.

In FIG. 1, a camera installed in the room 200 is illustrated as one aspect of the sensor 14. The camera is arranged to cover the eyes or arms of the person M (in particular, hands) in the field of view. The camera captures and outputs a motion image to the information processing device 10. Note that the camera may be installed in the terminal device 202.

The eye or arm movements of the person M can be sensed by analyzing the motion image captured by the camera. Specifically, when the person M is engaged in input work on the terminal device 202 as shown in FIG. 1, the analysis on the captured motion image allows measurement of the eye movements (e.g., ocular movements) of the person M directed to a display of the terminal device 202 or the movements of the hands (e.g., the manipulation speed) of the person M manipulating a keyboard of the terminal device 202.

Human's pulse rate can be measured by photo plethysmography using a light-emitting diode and an optical sensor (such as a photo transistor), for example.

Human's brainwaves can be sensed by near-infrared spectroscopy or electroencephalograph, for example. The near-infrared spectroscopy is an approach to observe variations in cerebral blood volume, using a light source and a light-receiving sensor. The electroencephalograph is a sensor that picks up and amplifies the small current generated through the activities in the brain from the electrodes on the skull to measure as brainwaves. The brainwave information includes data indicating underlying rhythms, including frequency bands such as α wave and β wave.

Examples of human's peripheral sites include the wrists, the fingers, the ears, and the nose. The temperatures of the peripheral sites can be measured by, for example, sensors that are attached to parts of the person's body.

The information processing device 10 obtains the biometric information of the person M sensed by the sensor 14. The information processing device 10 uses the obtained biometric information of the person M to determine the person M's conditions. The conditions of the person M include the person M's work efficiency and the person M's comfort. The “work efficiency” refers to the percentage of work the person M can do within a period of time. For example, when the person M is engaged in input work on the terminal device 202 as shown in FIG. 1, the work efficiency corresponds to a ratio of the actual amount of work (e.g., the amount of text entered, etc.) within a period of time to a standard amount of work that is feasible within the amount of time. The “comfort” means a quality of being pleasant, with no mental or physical discomfort. The comfort, as used herein, refers to pleasantness received from the sound environment.

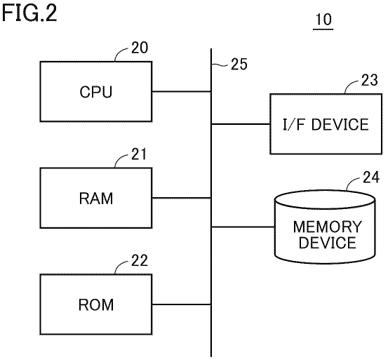

In the present embodiment, the information processing device 10 is configured to determine the person M's work efficiency, using the biometric information of the person M. As an example, the information processing device 10 can determine the person M's work efficiency from eye and/or hand movements of the person M within a period of time. In this case, the data indicating the relationship between the person M's eye and/or hand movements and the person M's work efficiency is previously obtained and stored in a memory device (see FIG. 2). The information processing device 10 refers to the data stored in the memory device to determine the person M's work efficiency, based on the person M's eye and/or hand movements within the period of time, which are sensed by the sensor 14.

Alternatively, the information processing device 10 can determine the person M's work efficiency from the person M's brainwaves within the period of time. Among the brainwaves, α wave a brainwave that often appears around the back of the head, generally, when a person is relaxed, such as during closed-eye resting. The β wave is a brainwave that often appears when a person is in wakefulness. Then, it is known that the alertness degree of a person can be estimated from the status of the brainwaves. The work efficiency deteriorates with a reduction in alertness degree. Thus, the alertness degree can be an index that indicates the work efficiency. In this case, data indicating the relationship between the person M's brainwaves and the person M's alertness degree within a period of time is previously obtained and stored in the memory device (see FIG. 2). The information processing device 10 refers to the data stored in the memory device to determine the person M's alertness degree, based on the person M's brainwaves within the period of time, which is sensed by the sensor 14.

The information processing device 10 is further configured to determine the person M's comfort, using the biometric information of the person M. For example, the information processing device 10 can determine the person M's comfort from the temperatures of the person M's peripheral body parts (such as the wrists, the fingers, the ears, and the nose). Generally, fluctuations in temperature of the peripheral body parts represent the thermoregulation conditions for each individual at a proper temperature, and are therefore used as an index that is suitable for the estimation of individual's comfort. The lower the temperatures of the peripheral body parts are, the lower the person's comfort tends to be.

Depending on the determined conditions of the person M, the information processing device 10 controls at least one of a component, frequency, and magnitude (sound pressure level) of the sound output from the output device 12. Specifically, depending on the person M's conditions, the information processing device 10 adjusts at least one of the frequency and magnitude (sound pressure level) of at least one frequency component forming the meaningless sound. The information processing device 10 also adjusts at least one of the frequency and magnitude (sound pressure level) of a frequency component forming the meaningful sound, depending on the person M's conditions. This causes the output device 12 to output into the room 200 the meaningful sound only, the meaningless sound only, or a synthetic sound obtained by synthesizing the meaningful sound and the meaningless sound. The output device 12 can further reproduce various meaningless sounds

### <Hardware Configuration of Information Processing Device>

FIG. 2 is a diagram showing a hardware configuration of the information processing device 10 of FIG. 1.

As shown in FIG. 2, the information processing device 10 includes a central processing unit (CPU) 20, a random access memory (RAM) 21, a read only memory (ROM) 22, an interface (I/F) device 23, and a memory device 24. The CPU 20, the RAM 21, the ROM 22, the I/F device 23, and the memory device 24 exchange various data therebetween through a communication bus 25.

The CPU 20 deploys a program stored in the ROM 22 into the RAM 21 and executes the program. Processes that are executed by the information processing device 10 are written in the program stored in the ROM 22.

The/F device 23 is an input/output device for exchange of signals and data with the output device 12 and the sensor 14. The I/F device 23 receives from the sensor 14 the biometric information of the person M sensed by the sensor 14. The/F device 23 also outputs a sound (an electrical signal) generated by the information processing device 10 to the output device 12.

The memory device 24 is a storage storing various information, including the biometric information of the person M, the information indicating the person M's conditions, and the data indicating the relationship between the biometric information of the person M and the person M's conditions. The memory device 24 is, for example, a hard disk drive (HDD) or a solid state drive (SSD).

### <Functional Configuration of Information Processing Device>

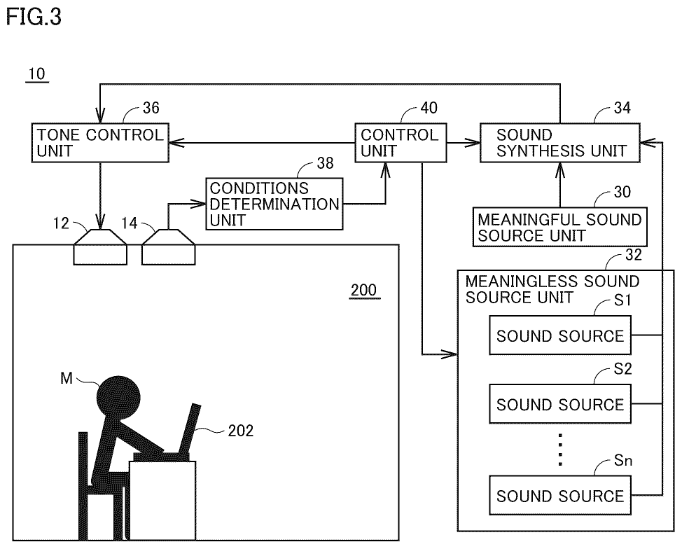

FIG. 3 is a diagram showing a functional configuration example of the information processing device 10. The functional configuration shown in FIG. 3 is implemented by the CPU 20 reading the program stored in the ROM 22, deploying the program to the RAM 21, and executing the program.

As shown in FIG. 3, the information processing device 10 includes a meaningful sound source unit 30, a meaningless sound source unit 32, a sound synthesis unit 34, a tone control unit 36, a conditions determination unit 38, and a control unit 40.

The meaningful sound source unit 30 is a sound source unit for generating the meaningful sound. As noted above, the meaningful sound is a sound that is meaningful to a person, typically, music. The meaningful sound source unit 30, for example, plays songs according to a play list defining the order of the songs to be played. Alternatively, the meaningful sound source unit 30 repeatedly plays previously-specified songs. The meaningful sound source unit 30 outputs the meaningful sound to the sound synthesis unit 34.

The meaningless sound source unit 32 is a sound source unit for generating the meaningless sound. The meaningless sound source unit 32 includes sound sources S1 through Sn (n is an integer greater than or equal to 2). The sound sources S1 through Sn are each configured to generate a sine wave (a sound wave) in the audio frequency band. The sine waves that are generated by the respective sound sources S1 through Sn have mutually different frequency components. The frequency of each sine wave varies temporally.

Specifically, a sound source Si (i is an integer greater than or equal to 1 and less than or equal to n) has an oscillator and is configured to generate a sine wave Xᵢ(t)=sin(2πfᵢ(t)*t) upon input of a frequency fᵢ(t). fᵢ(t) indicates that the frequency varies temporally. The meaningless sound source unit 32 adds up sine waves X₁(t) through Xₙ(t) to generate a synthetic wave of the sine waves. The meaningless sound source unit 32 outputs the synthetic wave to the sound synthesis unit 34.

The sound synthesis unit 34 is controlled by the control unit 40 and synthesizes the meaningful sound generated by the meaningful sound source unit 30 and the synthetic wave generated by the meaningless sound source unit 32. Here, a sound (a synthetic sound) Y(t) generated by the sound synthesis unit 34 can be represented by Equation (1) in a simplified manner.

$$Y ( t ) = {K}_{0} ( t ) * {X}_{0} + \sum ( {K}_{i} ( t ) * {X}_{i} ( t ) ) \qquad ( 1 )$$

Here, X₀ is the meaningful sound that is generated by the meaningful sound source unit 30. Xᵢ(t) is a sine wave generated by the sound source Si of the meaningless sound source unit 32. Kᵢ(t) is a coefficient whose value varies temporally, provided that i satisfies 1≤i≤n.

The second term on the right side of Equation (1) represents the meaningless sound generated by the meaningless sound source unit 32. The meaningless sound is generated by multiplying the sine waves X₁(t) through Xₙ(t) by the coefficients K₁(t) through Kₙ(t), respectively, and adding up the resultant values. The values of the coefficients K₁(t) through Kₙ(t) vary temporally, as described above. Varying the values of the coefficients K₁(t) through Kₙ(t) varies the amplitudes of the sine waves X₁(t) through Xₙ(t), respectively.

With this, at least one of the frequency and magnitude of the at least one frequency component forming the meaningless sound can be varied. Specifically, the frequency fᵢ(t) of the sine wave Xᵢ(t) varies temporally. The amplitude of the sine wave Xᵢ(t) varies temporally, depending on the value of the coefficient Kᵢ(t). As at least one of the frequency and amplitude of each of the sine waves X₁(t) through Xₙ(t) varies temporally, at least one of the frequency and magnitude of the at least one frequency component forming the meaningless sound varies. As a result, several types of meaningless sounds, including street sounds, the sound of running water of a river, can be reproduced.

As indicated in Equation (1), the synthetic sound is obtained by superimposing the meaningless sound on the meaningful sound. Adjusting the values of the coefficients K₀(t) through Kₙ(t) can vary the components of the synthetic sound. Note that the synthetic sound includes the meaningless sound only, if the coefficient K₀(t), which the meaningful sound X₀ is multiplied by, is set to zero. The synthetic sound includes the meaningful sound only, if the values of the coefficients K₁(t) through Kₙ(t), which the sine waves X₁(t) through Xₙ(t) are respectively multiplied by, are all set to zero, while the coefficient K₀(t) is set to a positive value. The sound synthesis unit 34 outputs the synthetic sound to the tone control unit 36.

The tone control unit 36 is controlled by the control unit 40 and adjusts at least one of the frequency and magnitude (sound pressure level) of the synthetic sound output from the output device 12. The tone control unit 36 is further configured to add a frequency component in the ultra-high frequency band (a frequency band higher than 20 kHz) to the synthetic sound. Note that, while individuals have different audio frequency bands, it is known that, in general, the frequency component in the ultra-high frequency band, which is the frequency band higher than 20 kHz, becomes more difficult for a person to hear with age. However, it has been found that α wave in the brainwaves increases as the frequency component of the ultra-high frequency band is transmitted to the brain through the skin and ear bones in the vicinity of the ears.

The conditions determination unit 38 obtains the biometric information of the person M sensed by the sensor 14. Using the obtained biometric information of the person M, the conditions determination unit 38 determines the person M's conditions. In the present embodiment, the conditions determination unit 38 uses the biometric information of the person M to determine the person M's work efficiency.

Specifically, the conditions determination unit 38 measures the person M's eye movements within a period of time from the motion image captured by the sensor 14 (e.g., the camera). The conditions determination unit 38 then refers to the data, stored in the memory device 24 (see FIG. 2), indicating the relationship between the person M's eye movements and the person M's work efficiency to calculate an index that represents the person M's work efficiency, based on measurements of the eye movements. The conditions determination unit 38 outputs the index to the control unit 40.

Based on the person M's work efficiency determined by the conditions determination unit 38, the control unit 40 controls the sound synthesis unit 34, the tone control unit 36, and the meaningless sound source unit 32. This allows the control unit 40 to vary the sound output from the output device 12 into the room 200, depending on the person M's work efficiency.

Specifically, the control unit 40 compares the index, representing the person M's work efficiency, provided by the conditions determination unit 38, with a predetermined threshold. Then, if the person M's work efficiency is lower than the threshold, the control unit 40 varies the components of the synthetic sound to be generated by the sound synthesis unit 34.

The synthetic sound is configured of the meaningful sound X₀, and the meaningless sound which consists of the sine waves X₁(t) through Xₙ(t) having mutually different frequency components, as indicated in Equation (1). The control unit 40 controls the sound synthesis unit 34 to vary the value of the coefficient K₀(t) which the meaningful sound X₀ is multiplied by, and at least one of the values of the coefficients K₁(t) through Kₙ(t) which the sine waves X₁(t) through Xₙ(t) are respectively multiplied by. Specifically, the control unit 40 varies the ratio of the meaningful sound to the synthetic sound by varying the value of the coefficient K₀(t). At this time, the meaningful sound can be removed from the synthetic sound if the coefficient K₀(t) is set to zero.

The control unit 40 also varies frequencies f₁(t) through fₙ(t) of the sine waves X₁(t) through Xₙ(t) and/or varies the values of the coefficients K₁(t) through Kₙ(t) to vary at least one of the frequency and magnitude (amplitude) of the at least one frequency component forming the meaningless sound to be included in the synthetic sound. With this, the type of the meaningless sound can be varied. For example, the control unit 40 may be configured to prepare multiple patterns corresponding to multiple types of meaningless sounds for the frequencies f₁(t) through fₙ(t) and the coefficients K₁(t) through Kₙ(t), and alternately select the patterns. Alternatively, the control unit 40 can remove the meaningless sound from the synthetic sound by setting all the values of the coefficients K₁(t) through Kₙ(t) to zero.

The control unit 40 further controls the tone control unit 36 to vary at least one of the frequency and magnitude (sound pressure level) of the synthetic sound adjusted by the sound synthesis unit 34. Varying the frequency of the synthetic sound varies the pitch of the synthetic sound. Specifically, the pitch of the synthetic sound increases with an increase of the frequency, and decreases with a decrease of the frequency.

The tone control unit 36 can adjust the magnitude of the sound at three levels: small; medium; and large, for example. Note that the tone control unit 36 may vary the frequencies and/or magnitudes of both the meaningful sound and the meaningless sound, or the frequency and/or magnitude of one of the meaningful sound and the meaningless sound. The tone control unit 36 can further add the frequency component of an ultra-high frequency band to the synthetic sound.

The control unit 40 controls the sound synthesis unit 34 and the tone control unit 36 as described above, while monitoring the person M's work efficiency provided by the conditions determination unit 38, to adjust at least one of the component, frequency, and magnitude of the synthetic sound output from the output device 12 into the room 200. At this time, the control unit 40 is configured to adjust at least one of the component, frequency, and magnitude of the synthetic sound so that the person M's work efficiency is greater than or equal to the threshold. Varying the sound environment in the room 200 depending on the person M's work efficiency in this manner enables restoration of the person M's work efficiency.

### <Sound Environment Control Method>

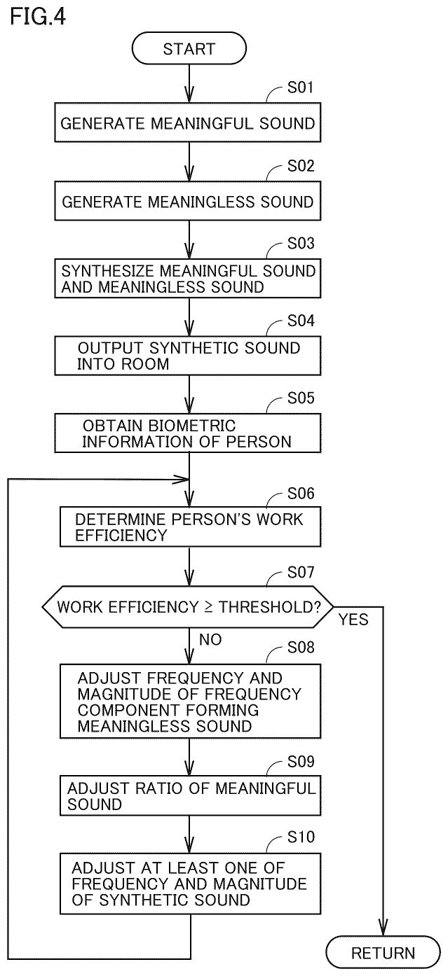

Next, a sound environment control method according to the present embodiment is described. FIG. 4 is a process flow diagram of the sound environment control method according to the present embodiment. The series of process steps illustrated in the flowchart are performed by the information processing device 10, for example, when a predetermined condition is met or for every predetermined cycle.

As shown in FIG. 4, the information processing device 10 generates the meaningful sound (step S01). In S01, the information processing device 10 plays songs, for example, according to a play list defining the order of the songs to be played. Alternatively, the information processing device 10 repeatedly plays previously-specified songs.

Subsequently, the information processing device 10 generates the meaningless sound (step S02). In S02, the information processing device 10 generates the sine waves X₁(t) through Xₙ(t) having mutually different frequency components, using the sound sources S1 through Sn. The frequencies f₁(t) through fₙ(t) of the sine waves X₁(t) through Xₙ(t), respectively, vary temporally. The information processing device 10 then adds up the sine waves X₁(t) through Xₙ(t), thereby generating a synthetic wave of the sine waves.

Subsequently, the information processing device 10 synthesizes the meaningful sound generated in S01 and the meaningless sound (a synthetic wave) generated in S02 (step S03). In S03, the synthetic sound is generated, using Equation (1) described above. Note that, as a default of the synthetic sound, a synthetic sound of a song and a previously-specified meaningless sound (e.g., the sound of crowds) may be set. In this case, the coefficient K₀(t), which the meaningful sound X₀ is multiplied by, is set to a positive value in Equation (1) and the values of the coefficients K₁(t) through Kₙ(t), which the sine waves X₁(t) through Xₙ(t) are respectively multiplied by, are set to patterns in which a previously-specified meaningless sound (e.g., the sound of crowds) is reproduced.

The information processing device 10 then transmits an electrical signal indicative of the synthetic sound generated in S03 to the output device 12. The output device 12 converts the electrical signal received from the information processing device 10 into an audio signal and outputs the audio signal into the room 200 as a sound (step S04). The sensor 14 senses the biometric information of the person M in the room 200. As an example, the sensor 14 is a camera installed in the room 200.

Next, the information processing device 10 obtains the biometric information of the person M sensed by the sensor 14 (step S05). In S05, as an example, the information processing device 10 measures the person M's eye movements within the period of time from the motion image captured by the camera as the sensor 14.

The information processing device 10, then, determines the person M's conditions, using the obtained biometric information of the person M (step S06) In S06, the information processing device 10 refers to the data, previously stored in the memory device 24 (see FIG. 2), indicating the relationship between the person M's eye movements and the person M's work efficiency to calculate an index representing the person M's work efficiency based on measurements of the eye movements.

Next, depending on the determined work efficiency of the person M, the information processing device 10 varies at least one of the component, frequency, and magnitude of the sound output from the output device 12 into the room 200.

Specifically, initially, the information processing device 10 compares the index representing the person M's work efficiency with the predetermined threshold (step S07). If the work efficiency is greater than or equal to the threshold (YES in S07), the information processing device 10 skips the subsequent process steps S08 through S10 to keep the sound output from the output device 12, thereby maintaining the sound environment in the room 200.

If the work efficiency is less than the threshold in step S07 (if NO in S07), in contrast, the information processing device 10 adjusts the frequency and magnitude (amplitude) of the at least one frequency component forming the meaningless sound that is included in the sound output from the output device 12 (step S08). In S08, the information processing device 10 varies the frequencies f₁(t) through fₙ(t) and/or the values of the coefficients K₁(t) through Kₙ(t) in Equation (1) to change the type of the meaningless sound. For example, the information processing device 10 can change the patterns of the frequencies f₁(t) through fₙ(t) and the coefficients K₁(t) through Kₙ(t) corresponding to the sound of crowds into the patterns of the frequencies f₁(t) through fₙ(t) and the coefficients K₁(t) through Kₙ(t) corresponding to other meaningless sound (e.g., nature sounds in a valley). Alternatively, the information processing device 10 can remove the meaningless sound from the sound output from the output device 12 by setting all the values of the coefficients K₁(t) through Kₙ(t) to zero.

Next, the information processing device 10 adjusts a ratio of the meaningful sound to the synthetic sound (step S09). In S09, the information processing device 10 varies the value of the coefficient K₀(t) in Equation (1) to vary the ratio of the meaningful sound to the synthetic sound. At this time, the information processing device 10 can remove the meaningful sound from the sound output from the output device 12 by setting the value of the coefficient K₀(t) to zero.

The information processing device 10 further adjusts at least one of the frequency and magnitude of the synthetic sound obtained by synthesizing the meaningless sound adjusted in S08 and the meaningful sound adjusted in S09 (step S10) In S10, the information processing device 10 may vary the frequencies of both the meaningful sound and the meaningless sound or vary the frequency of one of the meaningful sound and the meaningless sound. At this time, the information processing device 10 may add the frequency component of an ultra-high frequency band to the synthetic sound.

As the at least one of the component, frequency, and magnitude of the sound output from the output device 12 is varied through the process steps S08 through S10, the information processing device 10 returns to S06 and determines, again, the person M's work efficiency. Then, the information processing device 10 determines whether the determined work efficiency of the person M is greater than or equal to the threshold (step S07). If the work efficiency is improved to the threshold or greater (YES in S07), the information processing device 10 keeps the sound output from the output device 12, thereby maintaining the sound environment of the room 200. If the work efficiency is less than the threshold (NO in S07), in contrast, the information processing device 10 performs, again, the process steps S08 through S10 to vary the sound output into the room 200. The process steps S08 through S10 are repeatedly performed until the person M's work efficiency is greater than or equal to the threshold.

As described above, the sound environment control system 100 according to the present embodiment is configured to output into the room a synthetic sound of a meaningful sound that is meaningful to a person and a meaningless sound that is meaningless to a person. In the above configuration, the information processing device 10, then, adjusts the component of the synthetic sound, depending on a person's work efficiency determined from the biometric information of the person in the room. Specifically, the information processing device 10 can adjust at least one of the frequency and magnitude of at least one frequency component forming the meaningless sound to change the type of the meaningless sound. The information processing device 10 can also remove one of the meaningful sound and the meaningless sound from the synthetic sound. The information processing device 10 can further vary at least one of the frequency and magnitude of the synthetic sound output into the room, depending on the person's work efficiency. With this, since the sound environment in the room can be varied, depending on the work efficiency of a person in the room, person's work efficiency can be improved, independent of individual preferences.

### EXPERIMENTAL EXAMPLE

Next, experimental examples of a sound environment control which is performed using the sound environment control system 100 according to the present embodiment are described.

### Experimental Example 1

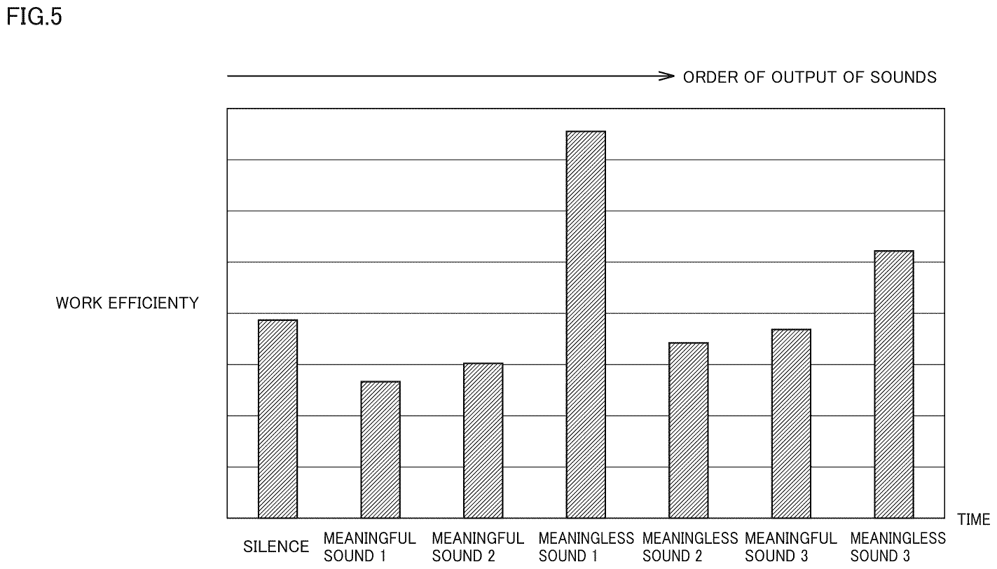

FIG. 5 is a graph showing a sound output from the sound environment control system 100 into the room versus the work efficiency of a subject in the room. In the graph, the horizontal axis indicates time, and the vertical axis indicates the subject's work efficiency. The subject is a healthy adult man.

In this experiment, the subject in the room was asked to engage in input work on a terminal device (a notebook), and images of the eye movements of the subject being engaged in the input work were captured by the sensor 14 (e.g., a camera). Then, in order for the information processing device 10 to determine the subject's work efficiency, data indicating the relationship between the eye movements and work efficiency of the subject was previously obtained and stored in the memory device 24 within the information processing device 10.

The graph of FIG. 5 shows variations in work efficiency of the subject with a temporally varying sound which was output from the output device 12 of the sound environment control system 100 into the room. As shown in FIG. 5, in the experiment, the sound output from the output device 12 into the room 200 was varied at predetermined time intervals, in order starting from a silence state to a meaningful sound 1 (e.g., a subject's favorite song), a meaningful sound 2 (e.g., a song the subject dislikes), a meaningless sound 1 (e.g., nature sounds in a valley), a meaningless sound 2 (e.g., automobiles driving), a meaningful sound 3 (e.g., reading aloud), and a meaningless sound 3 (e.g., the sound of crowds). All of these sounds were at the same magnitude (sound pressure level).

The meaningful sounds 1 to 3 were generated by the information processing device 10 setting the coefficient K₀(t), which the meaningful sound was multiplied by, to a positive value and setting the values of the coefficients K₁(t) through K₀(t), which the sine waves X₁(t) through Xₙ(t) were respectively multiplied by, to zero. The meaningless sounds 1 to 3 were reproduced by the information processing device 10 setting the value of the coefficient K₀(t) to zero and varying the frequencies of the sine waves X₁(t) through Xₙ(t) and/or the values of the coefficients K₁(t) through Kₙ(t) which the sine waves X₁(t) through Xₙ(t) were respectively multiplied by.

The information processing device 10 analyzed the motion image captured by the sensor 14 and thereby measured the eye movements of the subject within a period of time. The information processing device 10 then referred to the data stored in the memory device 24 to calculate an index representing the subject's work efficiency based on measurements.

As can be seen from the graph of FIG. 5, the subject's work efficiency varies with the sound environment of the room. In particular, it can be seen that the subject's work efficiency varies not only with the meaningful sound like a song and reading aloud, but also with the meaningless sound like nature sounds, automobiles driving, and the sound of crowds. In the experimental example of FIG. 5, it was confirmed that the work efficiency was high when the meaningless sounds 1 and 3 were output into the room, as compared to when the meaningful sound was output into the room.

According to the experimental result of FIG. 5, it can be seen that the subject's work efficiency is controllable by varying the sound output from the output device 12 into the room. Accordingly, the sound environment control system 100 is configured to vary the sound output into the room, while monitoring the subject's work efficiency, and the reduction of the subject's work efficiency can thereby by inhibited.

### Experimental Example 2

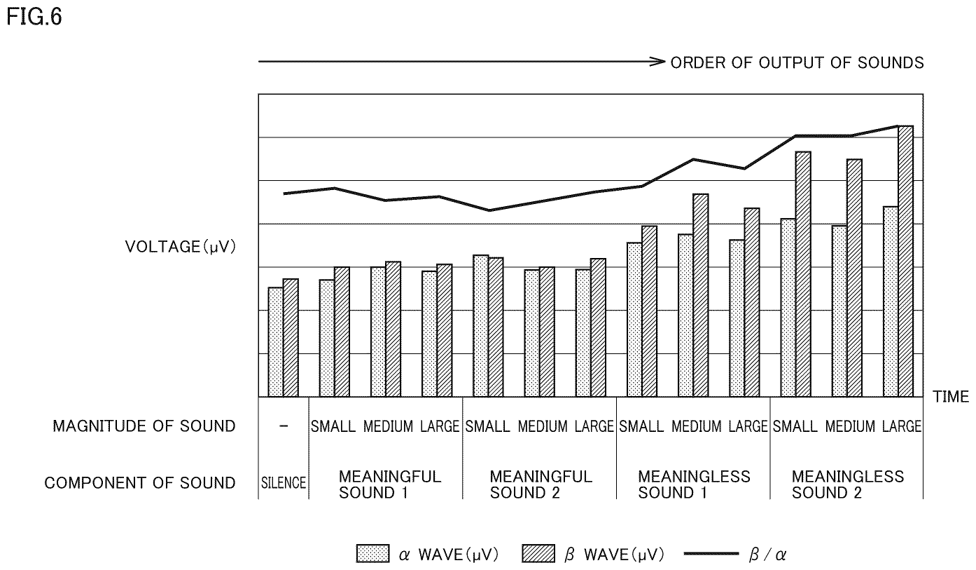

FIG. 6 is a graph showing a sound output from the sound environment control system 100 into the room versus a subject's brainwave state in the room. In the graph, the horizontal axis indicates time, and the vertical axis indicates the subject's brainwave state. The subject is a healthy adult man.

In this experiment, the subject in the room was asked to engage in input work on a terminal device (a notebook), and images of the subject's brainwaves were captured by the sensor 14 (e.g., an electroencephalograph) worn by the subject. Then, the information processing device 10 was used to determine the conditions (the alertness degree) of the subject, based on the brainwave information obtained from measurements from the sensor 14.

The graph of FIG. 6 shows variations in subject's brainwave state with a temporally varying sound which was output from the output device 12 of the sound environment control system 100 into the room. As shown in FIG. 6, in the experiment, the sound output from the output device 12 into the room was varied at predetermined time intervals, in order starting from a silence state to a meaningful sound 1 (e.g., an up tempo song), a meaningful sound 2 (e.g., classic music), a meaningless sound 1 (e.g., nature sounds in a valley), and a meaningless sound 2 (e.g., the sound of crowds). In this experimental example, each of the above four sounds were further varied in magnitude (sound pressure level) at three levels: small; medium; and large.

The meaningless sounds 1 and 2 were reproduced by the information processing device 10 setting the value of the coefficient K₀(t) to zero and varying the frequencies of the sine waves X₁(t) through Xₙ(t) and/or the values of the coefficients K₁(t) through Kₙ(t) which the sine waves X₁(t) through Xₙ(t) were respectively multiplied by.

The information processing device 10 detected the intensities of α wave and β wave included in the subject's brainwaves, based on the subject's brainwave information measured by the sensor 14 (an electroencephalograph). Note that the intensities of α wave and β wave are represented in voltage value (μV). The information processing device 10 further calculated the ratio of the intensity of β wave to the intensity of α wave (β/α) to estimate the alertness degree of the subject.

As can be seen from FIG. 6, the intensities of α wave and β wave included in the subject's brainwaves each vary with the sound environment in the room. In the meaningful sound 1 and the meaningful sound 2, the intensity of α wave and the intensity of β wave are at comparable levels. Note that any of the meaningful sounds are little varied in intensity of α wave and β wave due to the magnitude of the sound.

In addition, the meaningful sound 1 and the meaningful sound 2, although they have different tunes, did not show a significant difference therebetween both in intensity of α wave and in intensity of β wave. As a result, the ratio (β/α) varied little, too.

As the sound in the room varied from the meaningful sound 2 to the meaningless sound 1, in contrast, both α wave and β wave increased. In particular, a noticeable increase was seen in β wave. Due to the increase of β wave, the ratio (β/α) of the meaningless sound 1 increased, as compared to the meaningful sound 1 and the meaningful sound 2. Note that the variations in intensity of β wave also increased in the meaningless sound 1 relative to variations in magnitude of the sound.

Furthermore, as the sound in the room varied from the meaningless sound 1 to the meaningless sound 2, α wave and β wave further increased. In particular, a noticeable increase was seen in β wave. Due to the increase of β wave, the ratio (β/α) of the meaningless sound 2 has further increased, as compared to the meaningless sound 1. Similarly to the meaningless sound 1, the variations in intensity of β wave has also increased, relative to the magnitude of the sound.

Here, it is known that α wave increases when a person is relaxed, and β wave increases when a person is in wakefulness. In addition, the higher the ratio (β/α) is, the higher the alertness degree is. In the experimental example of FIG. 6, it was confirmed that α wave and β wave (in particular, R wave) increased and the ratio (β/α) is high in the meaningless sound 1 and the meaningless sound 2, as compared to the meaningful sound 1 and the meaningful sound 2. This suggests that the meaningless sound environment is more suitable for the subject to improve the alertness degree, as compared to the meaningful sound environment. In addition, it was confirmed that the intensity of β wave was controllable by the magnitude of the sound under the meaningless sound environment. With this, if the subject is determined to have a reduced alertness degree from the subject's brainwave information, it is expected that varying the sound environment of the room so that the sound environment control system 100 can let the subject listen to a meaningless sound enhances the alertness degree of the subject, thereby inhibiting the reduction of the subject's work efficiency.

### Other Configuration Examples

(1) In the above embodiment, depending on the work efficiency of a person in a room, which is determined from the biometric information of the person, the sound environment of the room is varied. However, the sound environment control system and the sound environment control method according to the present disclosure can also vary the sound environment of the room, depending on a person's comfort.

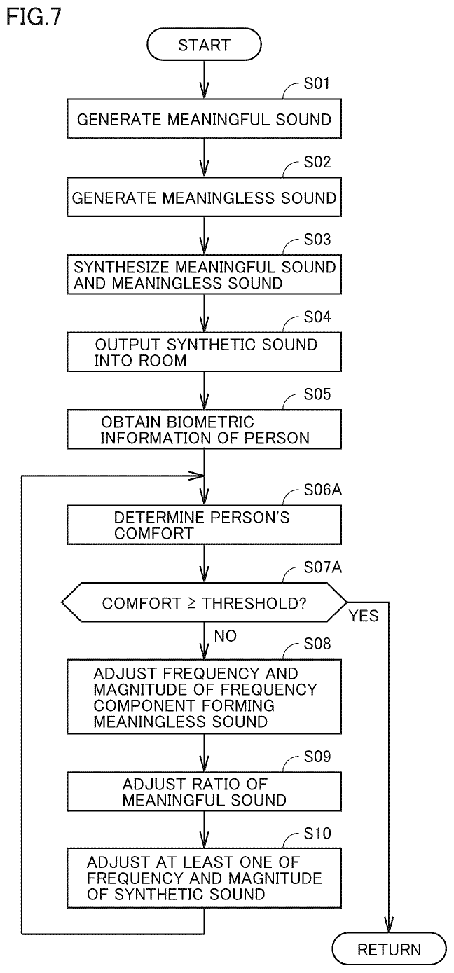

FIG. 7 is a process flow diagram of the sound environment control method according to Variation 1 of the present embodiment. The series of process steps illustrated in the flowchart are performed by the information processing device 10, for example, when a predetermined condition is met or for every predetermined cycle.

The flowchart of FIG. 7 includes S06A and S07A replacing S06 and S07 of the flowchart of FIG. 4. As illustrated in FIG. 7, by performing the same process steps S01 through S05 as those of FIG. 4, the information processing device 10 generates and outputs a synthetic sound of a meaningful sound and a meaningless sound into the room 200 via the output device 12 and obtains the biometric information of the person M sensed by the sensor 14. In S05, as an example, the information processing device 10 measures the temperature of peripheral body parts of the person M by the sensor 14 worn by the person M.

The information processing device 10, then, uses the obtained biometric information of the person M to determine the person M's conditions (step S06A) In S06A, the information processing device 10 refers to the data, previously stored in the memory device 24 (see FIG. 2), indicating the relationship between the temperatures of the peripheral body parts of the person M and the person M's comfort to calculate an index that represents the person M's comfort, based on measurements of the temperatures of the peripheral body parts.

Next, depending on the determined person M's comfort, the information processing device 10 varies at least one of the component, frequency, and magnitude of the sound output from the output device 12 into the room 200.

Specifically, initially, the information processing device 10 compares the index representing the person M's comfort with a predetermined threshold (step S07A). If the comfort is greater than or equal to the threshold (YES in S07A), the information processing device 10 skips the process steps S08 through S10 to keep the sound output from the output device 12, thereby maintaining the sound environment of the room 200.

If the comfort is less than the threshold in step S07A (NO in S07A), in contrast, the information processing device 10 performs the same process steps S08 through S10 as those of FIG. 4 to adjust the sound output from the room 200. At this time, the information processing device 10 repeats the process steps S08 through S10 until the person M's comfort is greater than or equal to the threshold.

As described above, the sound environment control system 100 according to Variation 1 of the present embodiment can also vary the sound environment of a room, depending on a person's comfort determined from the biometric information of the person in the room, thereby improving the person's comfort, independent of individual preferences.
(2) In the embodiment described above, the description is given with respect to the control of the sound environment when one person is present in a room. However, the sound environment control system and the sound environment control method according to the present disclosure are also applicable to cases where more than one person are present in the room.

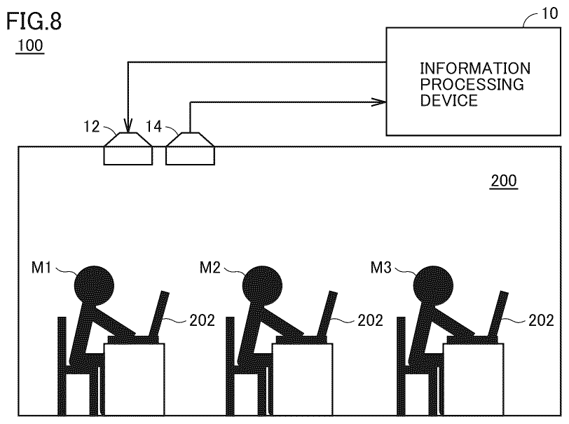

For example, as shown in FIG. 8, assume that multiple (e.g., three) persons M1 to M3 are present in the room 200. The persons M1 to M3 are all being engaged in input work on the terminal devices 202.

The sensor 14 senses the biometric information of the persons M1 to M3 in the room 200. The sensor 14 is, for example, a camera installed in the room 200, and arranged to cover the eyes or arms (in particular, hands) of the respective persons M1 to M3 in the field of view. The camera outputs a captured motion image to the information processing device 10. Note that the camera may be installed in the terminal device 202.

The information processing device 10 obtains the biometric information of the persons M t to M3 sensed by the sensor 14 and uses the obtained biometric information of the persons M1 to M3 to determine the conditions (e.g., work efficiencies) of the persons M1 to M3. Depending on the determined conditions (work efficiencies) of the persons M1 to M3, the information processing device 10 controls at least one of the component, frequency, and magnitude (sound pressure level) of the sound output from the output device 12.

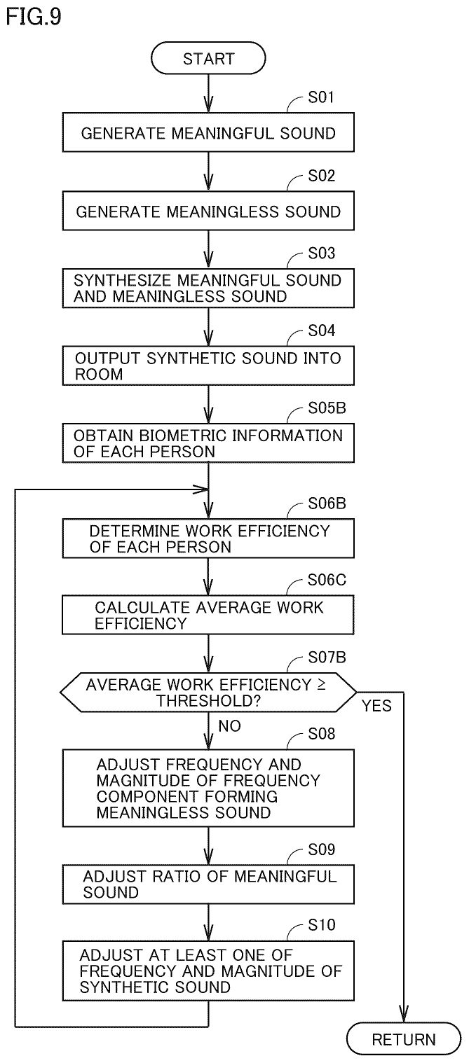

FIG. 9 is a process flow diagram of the sound environment control method according to Variation 2 of the present embodiment. The series of process steps illustrated in the flowchart are performed by the information processing device 10, for example, when a predetermined condition is met or for every predetermined cycle.

The flowchart of FIG. 9 includes S05B, S06B, S06C, and S07B replacing S05 through S07 of the flowchart of FIG. 4. As illustrated in FIG. 9, by performing the same process steps S01 through S04 as those of FIG. 4, the information processing device 10 generates and outputs a synthetic sound of a meaningful sound and a meaningless sound into the room 200 via the output device 12. The sensor 14 senses the biometric information of persons M1 to M3 in the room 200. As an example, the sensor 14 is a camera installed in the room 200.

Next, the information processing device 10 obtains the biometric information of the persons M1 to M3 sensed by the sensor 14 (step S05B). In S05B, as an example, the information processing device 10 measures the eye movements of the persons M1 to M3 within a period of time from motion images captured by a camera as the sensor 14.

The information processing device 10, then, uses the obtained biometric information of the persons M1 to M3 to determine the conditions of the persons M1 to M3, respectively (step S06B) In S06B, the information processing device 10 refers to the data, previously stored in the memory device 24 (see FIG. 2), indicating the relationship between the eye movements and work efficiency of each of the persons M1 to M3 to calculate an index representing the work efficiency of each of the persons M1 to M3, based on measurements of the eye movements.

Next, the information processing device 10 calculates an average of the work efficiencies of the persons M1 to M3 determined in S06B (step S06C). The information processing device 10, then, varies at least one of the component, frequency, and magnitude of the sound output from the output device 12 into the room 200, depending on the calculated average work efficiency.

Specifically, initially, the information processing device 10 compares the average work efficiency with a predetermined threshold (step S07B). If the average work efficiency is greater than or equal to the threshold (YES in S07B), the information processing device 10 skips the process steps S08 through S10 to keep the sound output from the output device 12, thereby maintaining the sound environment in the room 200.

If the average work efficiency is less than the threshold in step S07B (NO in S07B), in contrast, the information processing device 10 performs the same process steps S08 through S10 as those of FIG. 4 to adjust the sound output from the room 200. At this time, the information processing device 10 repeats the process steps S08 through S10 until the average work efficiency is greater than or equal to the threshold.

As described above, the sound environment control system 100 according to Variation 2 of the present embodiment can also vary the sound environment in a room, depending on people's work efficiencies determined from the biometric information of them in the room, thereby improving each person's work efficiency, independent of individual preferences.

The configuration example has been described in which the sound environment in the room 200 is varied if the average work efficiency of the persons M1 to M3 is less than the threshold (NO in S07B) in the flowchart of FIG. 9. However, the sound environment in the room 200 may be varied if at least one of the work efficiencies of the persons M1 to M3 is less than the threshold.

The presently disclosed embodiments should be considered in all aspects as illustrative and not restrictive. The technical scope of the present disclosure is defined by the appended claims, rather than by the description of the embodiments above. All changes which come within the meaning and range of equivalency of the appended claims are to be embraced within their scope.

### REFERENCE SIGNS LIST

10 information processing device; 12 output device; 14 sensor; 20 CPU; 22 ROM; 24 RAM; 26 I/F device; 28 memory device; 30 meaningful sound source unit; 32 meaningless sound source unit; 34 sound synthesis unit, 36 tone control unit; 38 conditions determination unit; 40 control unit; 100 sound environment control system; 200 room; 202 terminal device; M person; and S1 through Sn sound source.

1. A sound environment control system for controlling sound environment in a room in which a person is present, the sound environment control system comprising:
an information processing device to generate a sound having a plurality of frequency components;
an output device to output the sound generated by the information processing device into the room; and
a sensor to sense biometric information of the person, wherein
the plurality of frequency components include at least one frequency component in an audio frequency band, and
the sound includes a meaningless sound that is audible and meaningless to the person,
the information processing device:
determines conditions of the person, using the biometric information sensed by the sensor; and
adjusts at least one of a frequency and a magnitude of at least one frequency component forming the meaningless sound, depending on the determined condition of the person.

2. The sound environment control system according to claim 1, wherein
the conditions of the person include a work efficiency of the person, and
the information processing device:
determines the work efficiency, using the biometric information; and
varies, when the determined work efficiency decreases lower than a predetermined threshold, the at least one of the frequency and the magnitude of the at least one frequency component forming the meaningless sound so that the work efficiency increases greater than or equal to the threshold.

3. The sound environment control system according to claim 1, wherein
the conditions of the person includes comfort of the person, and
the information processing device:
determines the comfort, using the biometric information; and
varies, when the determined comfort decreases lower than a predetermined threshold, the at least one of the frequency and the magnitude of the at least one frequency component forming the meaningless sound so that the comfort increases greater than or equal to the threshold.

4. The sound environment control system according to claim 1, wherein
the information processing device is configured to form the meaningless sound by synthesizing a plurality of sine waves that have mutually different frequency components in an audio frequency band, and
the information processing device adjusts the at least one of the frequency and the magnitude of the at least one frequency component forming the meaningless sound by varying at least one of: frequencies; and amplitudes of the plurality of sine waves.

5. The sound environment control system according to claim 1, wherein
depending on the determined conditions of the person, the information processing device adds a frequency component in an ultra-high frequency band to the plurality of frequency components.

6. The sound environment control system according to claim 1, wherein
the sound further includes a meaningful sound that is meaningful to the person, and
the information processing device further adjusts a ratio of the meaningful sound to the sound, depending on the determined conditions of the person.

7. The sound environment control system according to claim 1, wherein
depending on the determined conditions of the person, the information processing device adjusts the at least one of the frequency and the magnitude of the sound output into the room.

8. The sound environment control system according to claim 1, wherein
the sensor senses eye movements of the person, movements of an arm of the person, and at least one of: a pulse rate; brainwaves; sweating; and temperatures of peripheral body parts of the person.

9. The sound environment control system according to claim 1, wherein
when a plurality of persons are present in the room,
the sensor senses the biometric information of the plurality of persons, and
the information processing device:
determines, for each person among the plurality of persons, the conditions of the person, using the biometric information sensed by the sensor; and
adjusts the at least one of the frequency and the magnitude of the at least one frequency component forming the meaningless sound, depending on an average of the determined conditions of the plurality of persons.

10. A sound environment control method for controlling sound environment in a room, comprising:
generating, by a computer, a sound having a plurality of frequency components;
outputting, by the computer, the generated sound into the room; and
sensing, using a sensor, biometric information of a person present in the room, wherein
the plurality of frequency components include at least one frequency component in an audio frequency band, and
the sound includes a meaningless sound that is audible and meaningless to a person, wherein
generating the sound includes:
determining conditions of the person, using the biometric information sensed by the sensor; and
adjusting at least one of a frequency and a magnitude of at least one frequency component forming the meaningless sound, depending on the determined conditions of the person.

11. The sound environment control method according to claim 10, wherein
the conditions of the person include a work efficiency of the person, and
the determining includes determining the work efficiency, using the biometric information, wherein
the adjusting includes varying, when the determined work efficiency decreases lower than a predetermined threshold, the at least one of the frequency and the magnitude of the at least one frequency component forming the meaningless sound so that the work efficiency increases greater than or equal to the threshold.

12. The sound environment control method according to claim 10, wherein
the conditions of the person includes comfort of the person, and
the determining includes determining the comfort, using the biometric information, and
the adjusting includes varying, when the determined comfort decreases lower than a predetermined threshold, the at least one of the frequency and the magnitude of the at least one frequency component forming the meaningless sound so that the comfort increases greater than or equal to the threshold.

13. The sound environment control method according to claim 10, wherein
generating the sound further includes forming the meaningless sound by synthesizing a plurality of sine waves that have mutually different the frequency components in an audio frequency band, and
the adjusting includes adjusting the at least one of the frequency and the magnitude of the at least one frequency component forming the meaningless sound by varying at least one of: frequencies; and amplitudes of the plurality of sine waves.

14. The sound environment control method according to claim 10, wherein
generating the sound further includes adding a frequency component in an ultra-high frequency band to the plurality of frequency components, depending on the determined conditions of the person.

15. The sound environment control method according to claim 10, wherein
the sound further includes a meaningful sound that is meaningful to the person, and
generating the sound further includes adjusting a ratio of the meaningful sound to the sound, depending on the determined conditions of the person.

16. The sound environment control method according to claim 10, wherein
generating the sound includes adjusting at least one of the frequency and the magnitude of the sound output into the room, depending on the determined conditions of the person.

17. A sound environment control system for controlling sound environment in a room in which a person is present, the sound environment control system comprising:
an information processing device to generate a sound having a plurality of frequency components;
an output device to output the sound generated by the information processing device into the room; and
a sensor to sense biometric information of the person, wherein
the sound includes a meaningless sound that is meaningless to the person, the information processing device:
determines conditions of the person, using the biometric information sensed by the sensor; and
adjusts at least one of a frequency and a magnitude of at least one frequency component forming the meaningless sound, depending on the determined condition of the person, wherein
when a plurality of persons are present in the room,
the sensor senses the biometric information of the plurality of persons, and
the information processing device:
determines, for each person among the plurality of persons, the conditions of the person, using the biometric information sensed by the sensor; and
adjusts the at least one of the frequency and the magnitude of the at least one frequency component forming the meaningless sound, depending on an average of the determined conditions of the plurality of persons.
